(19) United States
(12) Patent Application Publication
(10) Pub. No.: US 2007/0184085 A1
(43) Pub. Date: Aug. 9, 2007

(54) Ultrasound activated medical device

(75) Inventors: Rajesh Radhakrishnan, Bothell, WA (US); Chandru Chandrasekaran, Mercer Island, WA (US)

Correspondence Address: KENYON & KENYON LLP, 1500 K STREET N.W., SUITE 700, WASHINGTON, DC, 20005, US

(73) Assignee: BOSTON SCIENTIFIC SCIMED, INC.

(21) Appl. No.: 11/346,442

(22) Filed: Feb. 3, 2006

(51) Int. Cl. A61F 2/02

(52) U.S. Cl. 424/423

(57) ABSTRACT

A medical device comprising a medical device body having drug-loaded vesicles thereon. The vesicles are ultrasound sensitive and release the drug upon ultrasound stimulation. Also provided is a method for controlling drug release from a medical device using drug-loaded vesicles that are ultrasound sensitive.

### TECHNICAL FIELD

The present invention relates to drug-coated medical devices and methods of controlling drug release from the same.

### BACKGROUND OF THE INVENTION

Many implantable medical devices are coated with a drug or therapeutic agent that acts to improve the effectiveness of the device. One such example of a drug-coated implantable medical device is a stent. Stents are tubular structures formed in a mesh-like pattern that are designed to be inserted into an organ or vessel. For example, a coronary artery stent is placed in a coronary artery across an area of blockage after it has been opened by an angioplasty procedure. The stent serves as a permanent scaffolding for the newly widened coronary artery. In many instances, however, the stented vessel becomes blocked again (known as restenosis) due to various biological processes, including tissue healing and regeneration, scar formation, irritation, and immune reactions that lead to an excess proliferation of the cells. Therefore, many stents are coated with a drug, such as paclitaxel, that acts to inhibit the processes that cause restenosis.

It is desirable to control the rate of drug release from a drug-coated stent. Many stent coatings are formed of a polymer matrix into which the drug is dispersed. Because drug release is influenced by its rate of diffusion out of the polymer coating, most prior approaches to controlling drug release from a stent involve altering the composition of the polymer coating. In these prior approaches, the drug release kinetics of the stent is fixed by the particular drug release characteristics of the coating composition applied to the stent. In certain cases, however, physicians may wish to custom tailor drug release from a stent according to the needs of an individual patient. The optimal treatment regimen to prevent restenosis in one particular patient may require a different drug dosing, given at different time points, than another patient.

### SUMMARY OF THE INVENTION

In an embodiment, the present invention provides a medical device comprising a medical device body and a plurality of drug-containing vesicles disposed thereon. The plurality of drug-containing vesicles release the drug upon exposure to ultrasound energy.

In another embodiment, the present invention provides a method of controlling drug release from a medical device comprising the steps of providing a medical device comprising a medical device body having a plurality of drug-containing, ultrasound-sensitive vesicles disposed thereon, placing the medical device in a body of a patient, and exposing the vesicles on the medical device to ultrasound energy to release the drug.

### BRIEF DESCRIPTION OF THE DRAWINGS

The present invention will become more fully understood from the detailed description given herein and the accompanying drawings which are given for illustration only and do not limit the present invention.

FIG. 1 is a schematic illustration of a micelle.

FIG. 2 is a cross-sectional side view of a fragmentary portion of a medical device according to an embodiment of the present invention.

FIG. 3 is a cross-sectional side view of a fragmentary portion of a medical device according to another embodiment.

FIG. 4 is a graph illustrating the rate of drug release over time from the medical device shown in FIG. 2.

FIG. 5 is a cross-sectional side view of a fragmentary portion of a medical device according to another embodiment.

FIG. 6 is a graph illustrating the rate of drug release over time from the medical device shown in FIG. 5.

FIG. 7 is a cross-sectional side view of a fragmentary portion of a medical device according to another embodiment.

FIG. 8 is a cross-sectional side view of a fragmentary portion of a medical device according to another embodiment.

FIG. 9 is a graph illustrating the rate of drug release over time from the medical device shown in FIG. 8.

FIG. 10 is a cross-sectional side view of a fragmentary portion of a medical device according to another embodiment (showing the full depth of the medical device body to illustrate the through-openings).

FIG. 11 is a cross-sectional side view of a fragmentary portion of a medical device according to another embodiment (showing the full depth of the medical device body to illustrate the through-openings).

FIG. 12 is a cross-sectional side view of a fragmentary portion of a medical device according to another embodiment (showing the full depth of the medical device body to illustrate the through-openings).

FIG. 13 is a cross-sectional side view of a fragmentary portion of a medical device according to another embodiment.

FIG. 14 is a cross-sectional side view of a fragmentary portion of a medical device according to another embodiment.

FIG. 15 is a cross-sectional side view of a fragmentary portion of a medical device according to another embodiment.

### DETAILED DESCRIPTION OF THE INVENTION

The present invention provides a medical device comprising a medical device body having a plurality of drug-containing vesicles disposed thereon (unless otherwise indicated, the terms “drug” and “therapeutic agent” are used interchangeably herein). According to the present invention, the vesicles are ultrasound-sensitive drug carriers that release the drug contained therein when exposed to ultrasound energy. The vesicles have sufficient structural stability to retain the drug contained therein under non-exposed conditions (i.e., when not exposed to ultrasound energy) yet are able to become destabilized and release the retained drug upon exposure to ultrasound energy. The vesicles can be any type of carrier that can retain a drug such as, for example, a micelle, liposome, nanoparticle, bubble, microbubble, microsphere, microcapsule, clathrate bound vesicle, or hexagonal H II phase structure and can be manufactured of any ultrasonic-sensitive material such as, for example, ultrasound-sensitive lipids, proteinaceous materials, polymeric materials, carbohydrates, or surfactants. The vesicles can be fabricated from natural, synthetic, or semi-synthetic materials.

Vesicles of the present invention can have one or more membranes which define one or more voids. For example, the vesicles may have monolayers or multilayers, such as bilayers or trilayers. If vesicles have more than one membrane, such membranes can be concentric. The membranes can be substantially solid, porous, or semi-porous. Vesicles used in the present invention are preferably spherical in shape and are appropriately sized to serve as drug carriers, preferably with a radii in the range of 2 nm to 30 nm. However, other shapes and sizes are possible within the scope of the invention.

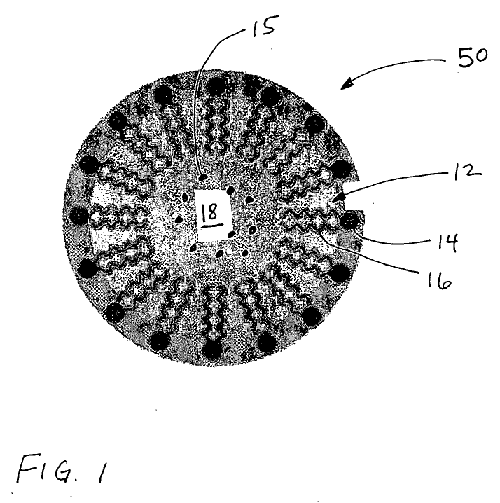

Referring to FIG. 1, a vesicle of the present invention may be a micelle 50. Micelles can be formed of amphiphilic molecules 12 having a polar hydrophilic terminal group 14 attached to a hydrophobic hydrocarbon chain 16. In an aqueous solution, amphiphilic molecules 12 form a spherical aggregate in which the hydrophilic polar head 14 of the molecules are exposed to the aqueous external environment and the hydrophobic tails 16 form a core 18 of micelle 50. Therapeutic agents 15 may be introduced into micelle core 18 by methods well known in the art, such as mixing the drug in a solution with the micelle-forming amphiphilic molecules 12 and then facilitating aggregation and drug encapsulation by sonication of the solution.

Further, micelle 50 may be fabricated from ultrasound-sensitive materials such as Pluronic P-105 triblock polymers as described in U.S. Pat. No. 6,649,702 to Rapoport et al., which is incorporated by reference herein. These polymeric micelles may be stabilized in various ways to serve as effective drug delivery carriers and to prevent degradation upon dilution in body fluids. Such stabilization methods include direct radical cross-linking of micelle cores, introduction of low concentrations of vegetable oil, or polymerization of temperature-responsive low critical solution temperature (LCST) hydrogel in the micelle cores. Moreover, these Pluronic P-105 triblock micelles are capable of releasing the drug when exposed to ultrasound energy. Without being bound by theory, it is thought that this drug release effect results from ultrasound-induced drug diffusion out of the micelles, or from micelle perturbation when acoustic shock waves cause transient cavitation, disrupting the micelles and allowing the drugs to escape.

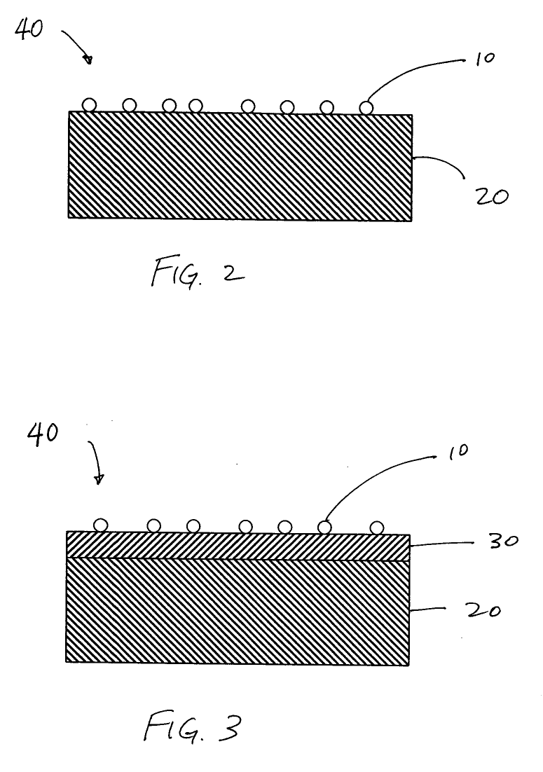

Referring to FIGS. 2 and 3, in certain embodiments of the present invention, drug-containing vesicles 10 may be disposed directly or indirectly on the body of a medical device 40. As shown in FIG. 2, medical device 40 can comprise a medical device body 20 and vesicles 10 disposed directly onto the outer surface of medical device body 20. Alternatively, as shown in FIG. 3, medical device 40 can comprise medical device body 20, a coating layer 30 disposed on the medical device body 20, and drug-containing vesicles 10 disposed on the surface of coating layer 30.

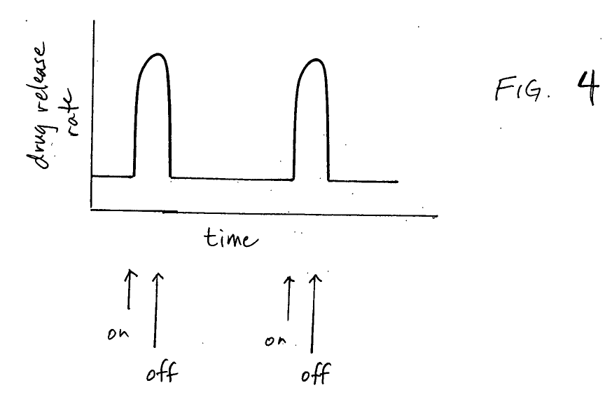

Vesicles 10 can be applied to the outer surface of medical device body 20 or outer surface of coating layer 30 by any method known in the art, such as spray coating, roll coating, or dip coating with a vesicle coating solution. Referring to the drug release profile shown in FIG. 4, because vesicles 10 are on an outer surface of medical device body 20 or coating 30, drug released from vesicles 10 can pass immediately into the external environment (i.e., the surrounding fluid or tissue), resulting in a sharp rise in the drug release rate. When the ultrasound stimulation ceases, vesicles 10 can revert to a stable, drug-retaining condition that seals any unreleased drug in vesicles 10.

As shown in FIG. 4 and as can be applied to other embodiments of the present invention, the release of drug is controlled in an on/off fashion corresponding to the duration of the ultrasound pulse (shown in the graph by the arrows indicating the ultrasound on/off points). If the drug has not been depleted from the vesicles, a repeat pulse of ultrasound energy at a later time triggers the release of another dose of drug (shown in the graph by the second surge of drug release). Alternatively, in other embodiments, vesicles do not revert to a stable, drug-retaining condition after cessation of ultrasound exposure. Rather, vesicles are permanently destabilized and there is continued release of drug even after ultrasound stimulation ceases. Further, in some embodiments, the vesicles completely entrap the drug until release is desired. Alternatively, in other embodiments, the vesicles do not completely entrap the drug and there is some continued release of drug in the absence of ultrasound stimulation. In such embodiments, ultrasound stimulation enhances the rate of drug release above a baseline level.

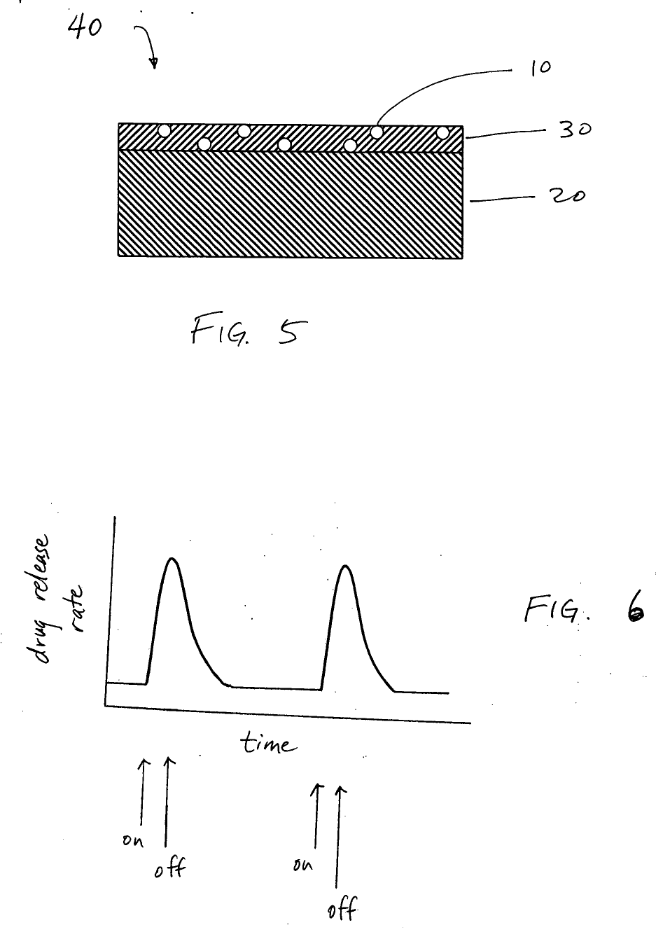

Referring to FIG. 5, in certain embodiments, medical device 40 comprises a medical device body 20 having a coating 30 disposed thereon and drug-containing vesicles 10 incorporated within coating 30. In one embodiment, coating 30 is a polymer layer with vesicles 10 embedded in the matrix of the polymer. Vesicles 10 may be incorporated into the polymer layer by mixing drug-containing vesicles 10 with the polymer solution and applying the mixture onto medical device 20 by any coating method known in the art, such as spraying or dip coating. Upon ultrasound stimulation, drug is released from vesicles 10 and instead of passing directly into the external environment, the drug first diffuses through the polymer matrix. Referring to the drug release profile shown in FIG. 6, this embodiment has a biphasic drug release profile that is typical of matrix-controlled drug release mechanisms. Vesicles 10 on or closest to the surface of the polymer layer will release drug directly into the surrounding fluid or tissue. Drug released from vesicles 10 deeper in the polymer layer requires a longer diffusion time. Thus, there is an initial burst release of drug followed by a progressive decrease in the rate of drug diffusion.

Referring again to FIG. 5, in another embodiment, coating 30 may be formed of a porous metallic or metallic oxide layer having a network of pores. Examples of metals that can be used to form this metallic layer include iridium, titanium, or chromium, and their metal oxides. This porous metallic or metallic oxide layer can be applied to medical device body 20 by various coating or deposition methods known in the art, such as electroplating, spray coating, dip coating, sputtering, chemical vapor deposition, or physical vapor deposition. Because drug deeper in the porous network requires a longer diffusion time than drug located closer to the surface, the drug release profile of this embodiment is similar to that shown in FIG. 6.

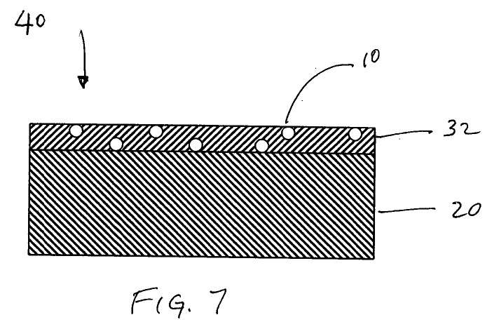

Referring to FIG. 7, in an alternate embodiment, medical device 40 comprises a medical device body 20 having a porous surface 32. Porous surface 32 can be created on medical device body 20 by treating the surface of medical device body 20 with micro-roughening processes such as reactive plasma treatment, ion bombardment, or micro-etching. Drug-containing vesicles 10 can be embedded within porous surface 32 by various methods, including spray coating, dip coating, vacuum impregnation, or electrophoretic transfer. The drug release kinetics of this embodiment is similar to that shown in FIG. 6. There is a biphasic drug release profile with an initial burst release of drug upon ultrasound stimulation, followed by a progressive decrease in the rate as drug deeper within the network of pores requires a longer diffusion time.

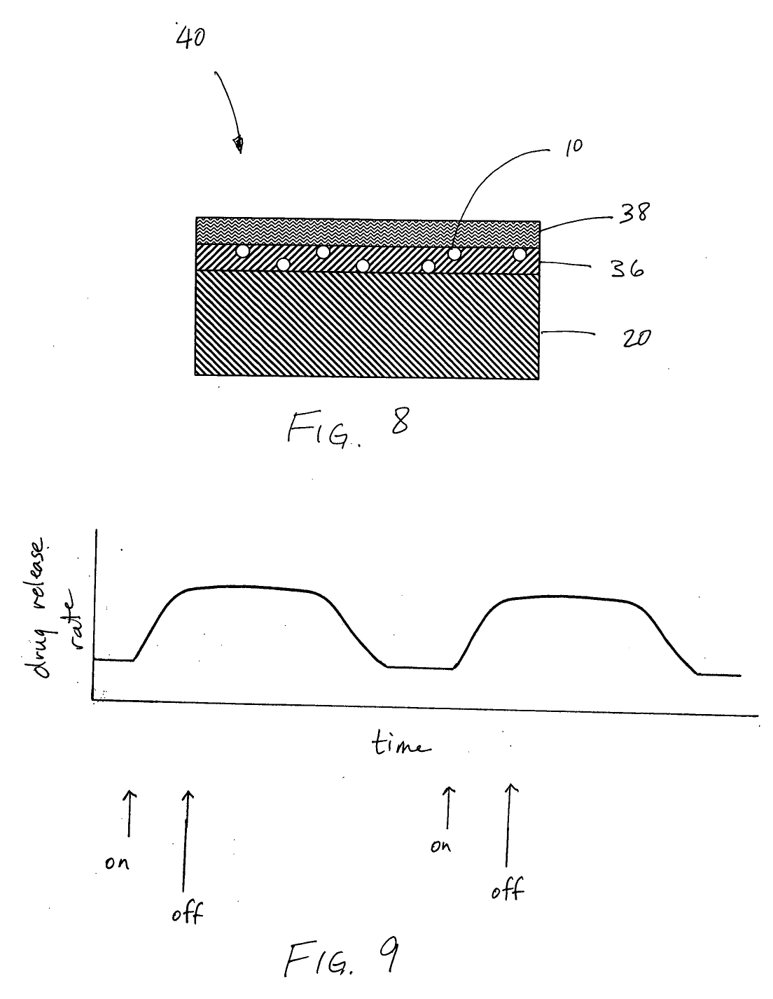

Referring to FIG. 8, in other embodiments, medical device 40 comprises a medical device body 20 having a reservoir layer 36 disposed thereon. Drug-containing vesicles 10 are incorporated within reservoir layer 36 and a semi-permeable barrier layer 38 is disposed on reservoir layer 36. Reservoir layer 36 can be any of the vesicle-containing layers described in any of the embodiments of the present invention. In these embodiments where medical device 40 comprises reservoir layer 36, medical device 40 constitutes a reservoir diffusion system of controlled drug release that is well known in the art. A reservoir diffusion system is designed so that a high concentration reservoir of drug is separated from the external environment by a semi-permeable barrier which limits the passage rate of drug molecules. Because the drug diffusion rate is restricted, once the drug concentration exceeds a critical level needed to meet the maximum diffusion capacity of the barrier, the drug release rate is constant over time until the drug concentration falls below a critical level.

In such embodiments, upon ultrasound activation, drug is released from vesicles 10 into reservoir layer 36, creating a concentrated reservoir of drug within the reservoir layer 36. Barrier layer 38 acts as a rate-limiting barrier limiting the rate at which drug diffuses out of reservoir layer 36 into the surrounding fluid or tissue. With continued ultrasound stimulation, the drug concentration in reservoir layer 36 exceeds a critical level where the diffusion rate through barrier layer 38 is at a maximum. As shown in FIG. 9, which represents the drug release kinetics of these embodiments upon on/off ultrasound stimulation, there is a constant rate of drug release from the stent, even after ultrasound stimulation has ceased. This constant drug release rate continues until the drug concentration in reservoir layer 36 falls below the critical level required to meet the maximum diffusion capacity of barrier layer 38. Barrier layer 38 can comprise any semi-permeable material such as drug-permeable polymers.

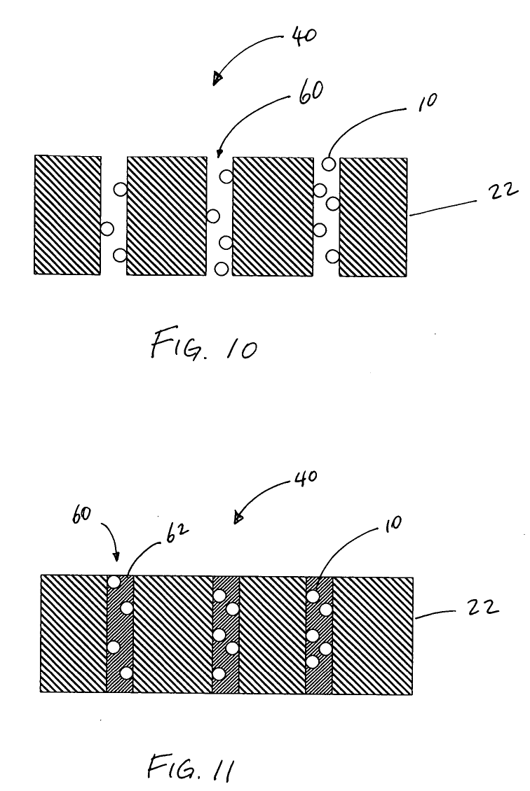

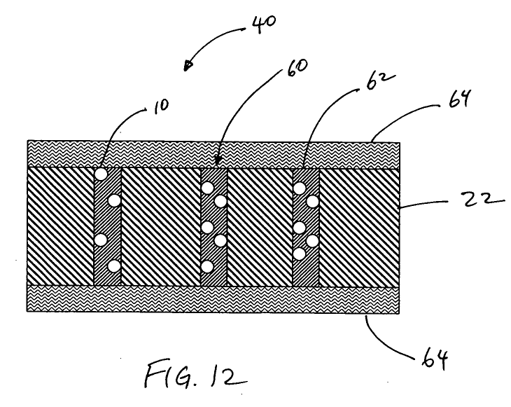

In other alternate embodiments, the body of the medical device may have vesicle reservoirs into which the vesicles are loaded, such. as the reservoirs described in U.S. Application Publication No. 2003/0199970, which is incorporated by reference herein. Referring to FIG. 10, in one such alternate embodiment, medical device 40 comprises a medical device body 22 having one or more through-openings 60. Through-openings 60 may be formed by laser drilling, electromachining, chemical etching, or any other means known in the art. Through-openings 60 are loaded with drug-containing vesicles 10. As shown in FIG. 11, through-openings 60 may further be loaded with a filler material 62 such as a polymer matrix. As shown in FIG. 12, the body of medical device 22 may be coated so that through-openings 60 are covered with a semi-permeable barrier layer 64. Filler material 62 and barrier layer 64 may be formed of the same or different materials and can be applied simultaneously or sequentially. This embodiment could function as a reservoir diffusion system such as the. one described for the embodiment of FIG. 8.

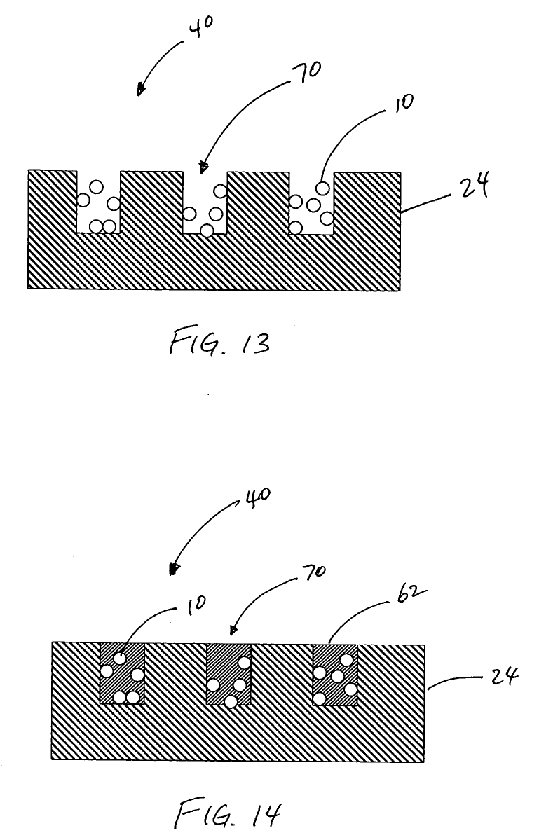

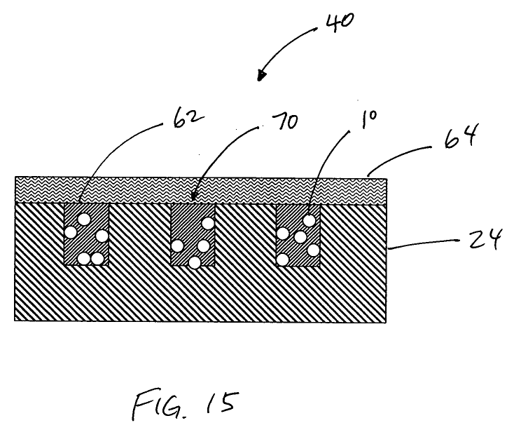

Referring to FIG. 13, in other alternate embodiments, the vesicle reservoirs may be recesses 70 instead of through-openings. Recesses 70 may be defined as grooves, pits, indentations, or any other openings in the surface of the medical device body 24 which do not extend through the entire depth of the medical device body. Recesses 70 may be formed by laser drilling, electromachining, chemical etching, or any other means known in the art. Recesses 70 are loaded with drug-containing vesicles 10. As shown in FIG. 14, recesses 70 may further be loaded with a filler material 62 such as a polymer matrix. As shown in FIG. 15, the body of medical device 24 may be coated so that recesses 70 are covered with a semi-permeable barrier layer 64. Filler material 62 and barrier layer 64 may be formed of the same or different materials and can be applied simultaneously or sequentially. This embodiment could function as a reservoir diffusion system such as the one described for the embodiment of FIG. 8.

The present invention also provides a method for controlling drug release from a medical device comprising the steps of: (1) providing a medical device comprising a medical device body having a plurality of drug-containing, ultrasound-sensitive vesicles thereon, (2) placing the medical device in a body of a patient and (3) exposing the plurality of vesicles to ultrasound energy to release the therapeutic agents. The ultrasound energy may be applied externally from the patient's body (e.g., transthoracic ultrasound) or internally (e.g., transesophageal, endoscopic, or intravascular ultrasound). The amount and duration of drug release from the vesicles is determined by various factors under the user's control, including the frequency, power density, and duration of the ultrasound exposure.

The medical devices of the present invention can be any medical device that can be used with the ultrasound-sensitive, drug-carrying vesicles, such as, for example, catheters, guide wires, balloons, filters (e.g., vena cava filters), stents, stent grafts, vascular grafts, intraluminal paving systems, pacemakers, electrodes, leads, defibrillators, joint and bone implants, vascular access ports, intra-aortic balloon pumps, heart valves, sutures, artificial hearts, neurological stimulators, cochlear implants, retinal implants, and other devices that can be used in connection with therapeutic coatings. Such medical devices can implanted or otherwise used in body structures such as the coronary vasculature, esophagus, trachea, colon, biliary tract, urinary tract, prostate, brain, lung, liver, heart, skeletal muscle, kidney, bladder, intestines, stomach, pancreas, ovary, uterus, cartilage, eye, bone, and the like.

The therapeutic agent in vesicles of the present invention may be any pharmaceutically acceptable agent such as a non-genetic therapeutic agent, a biomolecule, a small molecule, or cells.

Exemplary non-genetic therapeutic agents include anti-thrombogenic agents such heparin, heparin derivatives, prostaglandin (including micellar prostaglandin El), urokinase, and PPack (dextrophenylalanine proline arginine chloromethylketone); anti-proliferative agents such. as enoxaparin, angiopeptin, sirolimus (rapamycin), tacrolimus, everolimus, zotarolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis (2-aminoethyl) ethyleneglycol-N,N,N′,N′-tetraacetic acid and mixtures thereof, antibiotics such as gentamycin, rifampin, minocyclin, and ciprofloxacin; antibodies including chimeric antibodies and antibody fragments; anesthetic agents such as lidocaine, bupivacaine, and ropivacaine; nitric oxide; nitric oxide (NO) donors such as linsidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, warfarin sodium, Dicumarol, aspirin, prostaglandin inhibitors, platelet aggregation inhibitors such as cilostazol and tick antiplatelet factors; vascular cell growth promotors such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; inhibitors of heat shock proteins such as geldanamycin; angiotensin converting enzyme (ACE) inhibitors; beta-blockers; βAR kinase (βARK) inhibitors; phospholamban inhibitors; protein-bound particle drugs such as ABRAXANE™; and any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include serca-2 protein, monocyte chemoattractant proteins (MCP-1) and bone morphogenic proteins (“BMPs”), such as, for example, BMP-2, BMP-3, BMP-4, BMP-5, BMP-6 (VGR-1), BMP-7 (OP-1), BMP-8, BMP-9, BMP-10, BMP-11, BMP-12, BMP-13, BMP-14, BMP-15. Preferred BMPs are any of BMP-2, BMP-3, BMP-4, BMP-5, BMP-6, and BMP-7. These BMPs can be provided as homodimers, heterodimers, or combinations thereof, alone or together with other molecules. Alternatively, or in addition, molecules capable of inducing an upstream or downstream effect of a BMP can be provided. Such molecules include any of the “hedghog” proteins, or the DNA's encoding them. Non-limiting examples of genes include survival genes that protect against cell death, such as anti-apoptotic Bcl-2 family factors and Akt kinase; serca 2 gene; and combinations thereof. Non-limiting examples of angiogenic factors include acidic and basic fibroblast growth factors, vascular endothelial growth factor, epidermal growth factor, transforming growth factors α and β, platelet-derived endothelial growth factor, platelet-derived growth factor, tumor necrosis factor α, hepatocyte growth factor, and insulin-like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase and combinations thereof and other agents useful for interfering with cell proliferation.

Exemplary small molecules include hormones, nucleotides, amino acids, sugars, and lipids and compounds have a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or genetically engineered. Non-limiting examples of cells include side population (SP) cells, lineage negative (Lin−) cells including Lin⁻CD34⁻, Lin⁻CD34⁺, Lin⁻cKit⁺, mesenchymal stem cells including mesenchymal stem cells with 5-aza, cord blood cells, cardiac or other tissue derived stem cells, whole bone marrow, bone marrow mononuclear cells, endothelial progenitor cells, skeletal myoblasts or satellite cells, muscle derived cells, go cells, endothelial cells, adult cardiomyocytes, fibroblasts, smooth muscle cells, adult cardiac fibroblasts+5-aza, genetically modified cells, tissue engineered grafts, MyoD scar fibroblasts, pacing cells, embryonic stem cell clones, embryonic stem cells, fetal or neonatal cells, immunologically masked cells, and teratoma derived cells.

Any of the therapeutic agents may be combined to the extent such combination is biologically compatible. Further, each of the plurality of vesicles on the medical devices of the present invention can contain a single therapeutic agent or multiple therapeutic agents. Further, the plurality of vesicles can collectively contain the same therapeutic agents or at least some different therapeutic agents.

In embodiments of a medical device having a coating, such a coating can be biodegradable or non-biodegradable. Non-limiting examples of suitable non-biodegradable polymers include metals or metallic oxides; polystrene; polyisobutylene copolymers, styrene-isobutylene block copolymers such as styrene-isobutylene-styrene tri-block copolymers (SIBS) and other block copolymers such as styrene-ethylene/butylene-styrene (SEBS); polyvinylpyrrolidone including cross-linked polyvinylpyrrolidone; polyvinyl alcohols, copolymers of vinyl monomers such as EVA; polyvinyl ethers; polyvinyl aromatics; polyethylene oxides; polyesters including polyethylene terephthalate; polyamides; polyacrylamides; polyethers including polyether sulfone; polyalkylenes including polypropylene, polyethylene and high molecular weight polyethylene; polyurethanes; polycarbonates, silicones; siloxane polymers; cellulosic polymers such as cellulose acetate; polymer dispersions such as polyurethane dispersions (BAYHDROL®); squalene emulsions; and mixtures and copolymers of any of the foregoing.

Non-limiting examples of suitable biodegradable polymers include polycarboxylic acid, polyanhydrides including maleic anhydride polymers; polyorthoesters; poly-amino acids; polyethylene oxide; polyphosphazenes; polylactic acid, polyglycolic acid and copolymers and mixtures thereof such as poly(L-lactic acid) (PLLA), poly(D,L,-lactide), poly(lactic acid-co-glycolic acid), 50/50 (DL-lactide-co-glycolide); polydioxanone; polypropylene fumarate; polydepsipeptides; polycaprolactone and co-polymers and mixtures thereof such as poly(D,L-lactide-co-caprolactone) and polycaprolactone co-butylacrylate; polyhydroxybutyrate valerate and blends; polycarbonates such as tyrosine-derived polycarbonates and arylates, polyiminocarbonates, and polydimethyltrimethylcarbonates; cyanoacrylate; calcium phosphates; polyglycosaminoglycans; macromolecules such as polysaccharides (including hyaluronic acid; cellulose, and hydroxypropylmethyl cellulose; gelatin; starches; dextrans; alginates and derivatives thereof), proteins and polypeptides; and mixtures and copolymers of any of the foregoing. The biodegradable polymer may also be a surface erodable polymer such as polyhydroxybutyrate and its copolymers, polycaprolactone, polyanhydrides (both crystalline and amorphous), maleic anhydride copolymers, and zinc-calcium phosphate.

The medical devices of the present invention can comprise multiple layers of a coating that can be manufactured from the same or different material. Further, different layers can have vesicles containing different therapeutic agents or the same therapeutic agents. Further, therapeutic agents may be dispersed within the polymer coating itself, in addition to being loaded into vesicles.

A medical device of the present invention may also contain a radio-opacifying agent within its structure to facilitate viewing the medical device during insertion and at any point while the device is implanted. Non-limiting examples of radio-opacifying agents are bismuth subcarbonate, bismuth oxychloride, bismuth trioxide, barium sulfate, tungsten, and mixtures thereof.

The foregoing description and examples have been set forth merely to illustrate the invention and are not intended to be limiting. Each of the disclosed aspects and embodiments of the present invention may be considered individually or in combination with other aspects, embodiments, and variations of the invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

What is claimed is:

1. A medical device, comprising:
(a) a medical device body; and
(b) a plurality of vesicles disposed on the medical device body, wherein the plurality of vesicles contain therapeutic agents, and wherein the plurality of vesicles release the therapeutic agents when exposed to ultrasound energy.

2. The medical device of claim 1, wherein the vesicles are micelles.

3. The medical device of claim 2, wherein the micelles comprise amphiphilic block copolymers.

4. The medical device of claim 1, wherein the vesicles are disposed on the outer surface of a coating that coats the medical device body.

5. The medical device of claim 4, wherein the coating is a polymer coating.

6. The medical device of claim 4, wherein the coating is a metallic or metallic oxide coating.

7. The medical device of claim 4, further comprising a semi-permeable barrier layer disposed on the coating.

8. The medical device of claim 7, wherein the barrier layer is a polymer coating.

9. The medical device of claim 1, wherein the vesicles are disposed within a coating that coats the medical device body.

10. The medical device of claim 9, wherein the coating is a polymer coating.

11. The medical device of claim 9, wherein the coating is a metallic or metallic oxide coating.

12. The medical device of claim 9, further comprising a semi-permeable barrier layer disposed on the coating.

13. The medical device of claim 12, wherein the barrier layer is polymer coating.

14. The medical device of claim 1, wherein a surface of the medical device body is porous.

15. The medical device of claim 14, wherein the vesicles are disposed within the pores of the porous surface of the medical device body.

16. The medical device of claim 14, further comprising a semi-permeable barrier layer disposed on the porous surface of the medical device body.

17. The medical device of claim 16, wherein the barrier layer is a polymer coating.

18. The medical device of claim 1, wherein the medical device body includes one or more reservoirs.

19. The medical device of claim 18, wherein the vesicles are disposed within the reservoirs in the medical device body.

20. The medical device of claim 18, further comprising a semi-permeable barrier layer disposed on the surface of the medical device body.

21. The medical device of claim 20, wherein the barrier layer is a polymer coating.

22. A method for controlling drug release from a medical device, comprising the steps of:
(a) providing the medical device of claim 1;
(b) placing the medical device into a body of a patient; and
(c) exposing the plurality of vesicles to ultrasound energy to release the therapeutic agents.

23. The method of claim 22, wherein the ultrasound energy is from a source external to the body of the patient.

24. The method of claim 22, wherein the ultrasound energy is from a source internal to the body of the patient.
